(19) United States
(12) Patent Application Publication
(10) Pub. No.: US 2014/0051839 A1
(43) Pub. Date: Feb. 20, 2014

(54) Process for Reduction and/or Removal of FXI and FXIa from Solutions Containing said Coagulation Factors

(71) Applicants: Petra Schultz, VIENNA (AT); Gerhard Gruber, Vienna (AT); Frederic Bal, Lingolsheim (FR); Frank Marks, Springe (DE); Stefan Winge, Stockholm (SE)

(72) Inventors: Petra Schultz, VIENNA (AT); Gerhard Gruber, Vienna (AT); Frederic Bal, Lingolsheim (FR); Frank Marks, Springe (DE); Stefan Winge, Stockholm (SE)

(73) Assignee: Octapharma AG, Lachen (CH)

(21) Appl. No.: 13/885,612

(22) PCT Filed: Nov. 16, 2011
(86) PCT No.: PCT/EP2011/070257

§ 371 Date: Oct. 30, 2013

(30) Foreign Application Priority Data
Nov. 16, 2010 (EP) 10191398.6

(51) Int. Cl. C07K 16/00

(52) U.S. Cl. CPC C07K 16/00
USPC 530/390.5

(57) ABSTRACT

A process for reduction and/or removal of FXI and FXIa from a source solution containing said coagulation factors and as main components immunoglobulins comprising the following steps:
a) contacting the FXI and/or FXIa containing solution with an affinity chromatographic gel wherein heparin or heparan is linked to the matrix material;
b) allowing adsorption of FXI and/or FXIa and
c) separation of the liquid deprived of FXI and/or FXIa from the adsorption media.

The present invention pertains to a process for reduction and/or removal of FXI and FXIa from solutions containing said coagulation factors, a concentrate containing FXI and FXIa obtainable by the process of the invention and a pharmaceutical composition containing FXI and FXIa obtainable by the process of the invention.

Coagulation factor XI (FXI) is well known to be a protein involved in the coagulation of blood and represents one part of the intrinsic pathway of the coagulation cascade. FXI is the precursor of activated FXI (FXIa), which is the active compound during coagulation. Therefore it is essential to remove FXIa from pharmaceutical preparations being intravenously applied to patients as said FXIa may unintentionally start coagulation leading to life endangering thrombotic events. A concentrate of FXI and/or FXIa might on the other hand be beneficial for patients suffering from a disease related to lack or insufficient activity of FXI or for patients experiencing heavy blood loss where fast and effective coagulation is vitally important. Such concentrate can also be beneficial for patients suffering from inhibitory antibodies to coagulation factors, such as in Haemophilia A and B. Those inhibitors can cause bleeding events, thus patients require agents to support coagulation and wound closure.

It is thus an objective of the present invention to remove or at least reduce FXI and/or FXIa from solutions which contain FXI and/or FXIa. Another objective of this invention is to provide a therapeutically applicable concentrate of FXI, FXIa or a therapeutically applicable concentrate comprising a mixture of FXI and FXIa by the method presented.

Several methods to prepare a FXI concentrate are known. Bouma and Griffin published in THE JOURNAL OF BIOLOGICAL CHEMISTRY, 1977, vol. 252, no. 18, pp. 6432-6437 a process for purification of FXI from human plasma by five chromatographic steps. The chromatographic materials used in a chronological order are DEAE-Sephadex®, QAE-Sephadex®, SP-Sephadex® (twice) and finally Concanavalin A bound to Sepharose® All buffers used contain Polybrene® and benzamidine.

M. BURNOUF-RADOSEVICH AND T. BURNOUF reported in TRANSFUSION, 1992, 32, pp. 861-867 the production of a FXI lyophilisate by filtration of cryo-poor plasma over a negatively charged filter (Zeta plus 50S), which adsorbs FXI, elution of the adsorbed FXI and chromatography of the eluate on a cation exchange resin (Sulfate-Sepharose® Fast Flow). Eluted FXI was formulated with antithrombin and heparin prior to lyophilisation.

Hiroshl Mashiko and Hidenobu Takahashi disclosed in BIOL. CHEM. HOPPESEYLER, 1994, vol. 375, pp. 481-484 a production method for porcine FXI and FXIa based on high molecular mass kininogen-affinity chromatography and chromatography on Q-Sepharose®. To avoid contact activation and overcome proteolytical digestion they added Polybrene® and benzamidine to the buffers used. They also reported that they failed to purify FXI by Heparin-Sepharose®-affinity chromatography.

Another paper of Hiroshl Mashiko and Hidenobu Takahashi discussed in AGENTS AND ACTIONS SUPPLEMENTS, (BIRKHAEUSER VERLAG, BASEL, CH), 1992, vol. 38, part 2, pp. 249-256 a production method for porcine FXI and FXIa based on chromatography with high molecular mass kininogen, Q-Sepharose® and Protein A-Superose with Polybrene and benzamidine present in buffers.

Tait and Fujikawa disclosed in JOURNAL OF BIOLOGICAL CHEMISTRY, 1981, vol. 262, no. 24, pp. 11651-11656 a method to purify FXI and prekallikrein from hu- man plasma by three chromatographic steps and the use of Polybrene® and benzamidine in the buffers. A synthetic peptide representing one part of the light chain of high molecular weight kininogen was immobilized on a carrier and used to isolate prekallikrein, FXI and some carryover of IgG from plasma. Prekallikrein and FXI were subsequently separated by heparin-agarose chromatography and finally polished by CM-Sephadex® to obtain apparently pure fractions of FXI and prekallikrein.

Saito et al. presented in THE JOURNAL OF CLINICAL INVESTIGATION, vol. 52, pp. 850-861, 1973 a purification method for FXI consisting of adsorption of plasma on Ca₃(PO₄)₂ , multiple ammonium sulfate fractionation, and successive chromatography on QAE-Sephadex (twice), Sephadex®-G150, and SP-Sephadex® with Polyprene® present to prevent activation of FXI.

### SUMMARY OF THE INVENTION

The present invention provides a process for reducing the content of FXI, FXIa or a mixture of both from a solution containing said proteins and as main component immunoglobulins. This is achieved by adsorption of said proteins on adsorbing material selected from silicates (in particular silica, perlites, zeolithes or diatomaceous earth), aluminium hydroxide (Al₂(OH)₃), aluminium oxide hydroxide (AlO(OH)), aluminium oxide (Al₂O₃) or materials suitable for affinity chromatography. Said materials suitable for affinity chromatography are composed of polysaccarides, e.g. dextrane, heparin or heparan, linked to matrix material (e.g. zeolithes or polymers such as acrlyates and saccarides), in particular a gel used for heparin affinity chromatography such as e.g. Heparin Sepharose™ FF or Toyopearl AF Heparin 650 M™.

The solution containing source may be any liquid containing FXI and/or FXIa derived from blood or blood plasma or liquids derived from biotechnological processes. Known but not limiting examples of such solutions are cryo-poor plasma, intermediates of the Cohn process (e.g. reconstituted paste I+II+III) and its derivatives, intermediates of the Kistler-Nitschmann process (e.g. reconstituted precipitate A) and its derivatives or solutions resulting from recombinant protein expression but also solutions which are primarily composed of other proteins, wherein FXI or FXIa represent an impurity, which may be the case for solutions of immunoglobulin-gamma (IgG).

The process for reduction and/or removal of FXI and FXIa from solutions containing said coagulation factors and as main component immunoglobulins comprises the following steps:
a) contacting the FXI and/or FXIa containing solution with an affinity chromatographic gel wherein heparin or heparan is linked to the matrix material;
b) allowing adsorption of FXI and/or FXIa and
c) separation of the liquid deprived of FXI and/or FXIa from the adsorption media.

It is also possible to modify step a) in so far that the active sites of the matrix or gel are either already saturated or are allowed to saturate with antithrombin during loading of the matrix or gel with FXI and/or FXIa.

Adsorption may either be performed as batch adsorption wherein the source solution is mixed with the adsorbent, stirred and FXI and/or FXIa loaded onto the adsorbent is/are removed from the supernatant by known processes like sedimentation, filtration or centrifugation. An alternative procedure is packing of adsorption material into a chromatographic column and application of the source solution to load the adsorbent with FXI and/or FXIa.

In an embodiment of the invention silicates selected from the group of silica, perlites, zeolithes or diatomaceous earth may be additionally used as adsorbens of FXI and/or FXIa.

In a further embodiment an additional adsorption medium selected from the group of aluminium hydroxide, aluminium oxide hydroxide or aluminium oxide may be used.

It is also possible to combine an adsorption preformed in batch modus for instance with diatomaceous earth or aluminum hydroxide as adsorption material with an adsorption performed in a chromatographic column with Heparin Sepharose™ as adsorbent.

In still another embodiment of the invention one adsorption on heparin or heparan linked to a matrix material is performed after the chromatographic material was preconditioned with Antithrombin-III.

Loaded adsorbent is carefully washed with a washing buffer to avoid desorption of FXI and/or FXIa and resulting wash solution may be added to the flow-through and/or a supernatant to be further processed and to optimize recovery of other compounds of interest, such as immunoglobulins, in particular IgG, in this FXI/FXIa-depleted solution. A FXI/FXIa-depleted solution processed over Heparin Sepharose™ typically contains less than 0.15 IU FXI/ml, in particular less than 0.1 IU FXI/ml even more particular from 0.00 to 0.05 IU FXI/ml. The content of FXIa expressed in international units (IU) of such a depleted solution is typically less than 10 mU FXIa/ml, in particular less than 5 mU FXIa/ml, even more particular from 0.0 to 1.0 mU FXIa/ml.

Further processing of the FXI/FXIa-depleted solution may incorporate one or more virus inactivation steps, examples given are solvent/detergent treatment (S/D treatment), UV-radiation, pasteurization, low pH incubation, caprylate precipitation or nanofiltration. Other steps include chromatographic steps, concentration to obtain a concentrate of a pharmaceutically active compound, formulation and filling, which are known from manufacturing of various proteins such as immunoglobulins, in particular IgG, albumin, fibrinogen, antithrombin or alpha-1-antitrypsin, and are mandatory in order to obtain pharmaceutical compositions and depend on the product to be produced.

FXI and/or FXIa may be eluted from the loaded adsorbent, in particular if the adsorbent is an affinity chromatography gel with heparin or heparan attached to the matrix. Elution of FXI and/or FXIa from an affinity chromatography gel is performed with an elution buffer consisting of 0.2-1.4 M NaCl, in particular 0.25-1.0 M NaCl, even more particular 0.25-0.5 M NaCl and 0.003-0.03 M phosphate or equivalent ion strength. Thus afforded solution containing FXI and/or FXIa can also be virus inactivated by methods mentioned above and concentrated by ultra/diafiltration to obtain a concentrate containing FXI, FXIa or both. Said concentrate may further be formulated with adjuvants to obtain a pharmaceutical composition capable of treating diseases related to lack or inactivity of FXI or FXIa.

It was surprising to find FXI and FXIa levels of the effluent of a heparin chromatography gel below the detection limit of said proteins although antithrombin was allowed to break through. It was expected that FXI and FXIa would have been replaced by antithrombin, in particular the antithrombin-β isoform, as it is known to strongly bind to heparin and heparan affinity gels. It was even more astonishing that it was possible to selectively elute FXI and FXIa from the antithrombin saturated heparin affinity gel with elution buffers of low to intermediate ionic strength, essentially containing 0.2-1.4 M NaCl or buffers of equivalent ionic strength, while it is necessary to elute antithrombin-III (AT-III) with buffers of higher ionic strength, essentially containing more than 1.5 M NaCl, in particular at about 2.0 M NaCl. This feature allows either separate elution of FXI/FXIa followed by elution of AT-III or co-elution of a mixture containing FXI, FXIa and AT-III when eluting with a buffer of sufficiently high ionic strength to also elute AT-III.

It was even more surprising to find unnecessary the addition of Polybrene and benzamidine to buffers, as generally taught by prior art literature to avoid contact activation and overcome proteolytical digestion.

One objective of the present invention is performed to the best advantage by preconditioning affinity chromatographic gels with dextrane, heparin or heparan linked to the matrix material with antithrombin-III (AT-III). Preconditioning may be performed to such an extent that the active sites of the chromatographic material are saturated with AT-III. This procedure is especially beneficial as the binding capacity of the affinity gel for FXI and FXIa is improved and binding of other coagulation factors is prohibited or at least hindered to a high degree. It is thus possible to remove FXI and FXIa selectively from accompanying proteins and obtain a solution of FXI and FXIa devoid of other coagulation factors after elution from the affinity gel.

A concentrate of a pharmaceutically active component obtainable by the process of the invention as well as a pharmaceutical composition obtained thereof is also subject matter of the invention.

In the concentrate of the pharmaceutically active component according to the invention or the pharmaceutical composition of the invention the pharmaceutically active component is IgG.

### DETAILED DESCRIPTION OF THE INVENTION

The present invention provides a process for reducing the content of FXI, FXIa or a mixture of both from a solution containing said proteins and as main component immunoglobulins. This is achieved by adsorption of said proteins on adsorbing material selected from silicates (in particular diatomaceous earth), or materials suitable for affinity chromatography, in particular a gel used for heparin- or heparan-affinity chromatography such as e.g. Heparin Sepharose™ FF or Toyopearl AF Heparin 650 M™.

The solution containing source may be any liquid containing FXI and/or FXIa derived from blood or blood plasma or liquids derived from biotechnological processes. Known but not limiting examples of such solutions are cryo-poor plasma, intermediates of the Cohn process (e.g. reconstituted paste I+II+III) and its derivatives, intermediates of the Kistler-Nitschmann process (e.g. reconstituted precipitate A) and its derivatives or solutions resulting from recombinant protein expression but also solutions which are primarily composed of other proteins, wherein FXI or FXIa represent an impurity, which may be the case for solutions of immunoglobulin-gamma (IgG).

The process for reduction and/or removal of FXI and FXIa from solutions containing said coagulation factors and as main component immunoglobulins comprises the following steps:
a) contacting the FXI and/or FXIa containing solution with an affinity chromatographic gel wherein heparin or heparan is linked to the matrix material;
b) allowing adsorption of FXI and/or FXIa and
c) separation of the liquid deprived of FXI and/or FXIa from the adsorption media.

It is also possible to modify step a) in so far that the active sites of the matrix or gel are either already saturated or are allowed to saturate with antithrombin during loading of the matrix or gel with FXI and/or FXIa.

An alternative procedure is packing of adsorption material into a chromatographic is column and application of the source solution to load the adsorbent with FXI and/or FXIa.

In an embodiment of the invention diatomaceous earth may be additionally used as adsorbens of FXI and/or FXIa.

Steps a) and b) are performed by loading the proteins in a buffer with a conductivity of 10-18 mS, in particular with a conductivity of 14-17 mS, onto the chromatographic resin (Heparin-Sepharose™ FF). Loaded adsorbent is carefully washed with a washing buffer of the same conductivity to avoid desorption of FXI and/or FXIa and resulting wash solution may be added to the flow-through and/or a supernatant to be further processed and to optimize recovery of IgG present in the flow-through as FXI/FXIa-depleted solution. The FXI/FXIa-depleted solution processed over Heparin Sepharose™ typically contains less than 0.1 IU FXI/ml even more particular from 0.00 to 0.05 IU FXI/ml. The content of FXIa expressed in international units (IU) of such a depleted solution is typically less than 5 mU FXIa/ml, even more particular from 0.0 to 1.0 mU FXIa/ml.

Further processing of the FXI/FXIa-depleted solution may incorporate one or more virus inactivation steps, examples given are solvent/detergent treatment (S/D treatment), as disclosed in EP-A-131 740 incorporated by reference, UV-radiation, pasteurization, low pH incubation, caprylate precipitation or nanofiltration. Other steps include chromatographic steps, concentration to obtain a concentrate of a pharmaceutically active compound, formulation and filling, which are known from manufacturing of various proteins such as immunoglobulins, in particular IgG, albumin, fibrinogen, antithrombin or alpha-1-antitrypsin, and are mandatory in order to obtain pharmaceutical compositions and depend on the product to be produced.

FXI and/or FXIa may be eluted from the loaded adsorbent with an elution buffer consisting of 0.36 M NaCl and 0.01 M phosphate or equivalent ion strength. Thus afforded solution containing FXI and/or FXIa can also be virus inactivated by methods mentioned above and concentrated by ultra/diafiltration to obtain a concentrate containing FXI, FXIa or both. Said concentrate may further be formulated with adjuvants to obtain a pharmaceutical composition capable of treating diseases related to lack or inactivity of FXI or FXIa.

### EXAMPLES

Factor XIa Activity Assay

A recombinant coagulation factor IX (void of FIXa) is activated to FIXa by FXIa present in the sample. In the presence of phospholipids and calcium ions FIXa forms an enzyme complex with thrombin-activated FVIII:C which is in excess in the assay solution. This enzyme complex subsequently activates FX, which is also present in the assay solution, to Factor Xa (FXa). The generated amount of FXa is measurable by commercially available substrates and direct proportional to the FXIa concentration in the sample. Quantification is done by comparison with a calibration curve.

It has to be mentioned that this assay also indicates the activities of FIXa and FXa when samples from early stages of the plasma fractionation process, such as cryo-poor plasma, are measured. It is thus comprehensible that the summarized activities of FXIa, FIXa and FXa are indicated for such samples, with the prerequisite that FIXa and Fxa are present in the sample.

### Example 1

Starting material was processed over Heparin Sepharose FF packed in a column to allow adsorption of FXI and FXIa on the chromatographic material. The IgG containing flow-through was brought in contact with Hyflo, i.e. diatomaceous earth, centrifuged to remove loaded Hyflo and the IgG containing supernatant was subsequently processed to the intermediate paste I+II+III. Reconstitution of thus produced intermediate revealed 0.02 IU FXI/ml and less than 1mU FXIa/ml.

### Example 2

Paste I+II+III was produced in the same way as example 1 with the exemption of omitting the FXI/FXIa-capture on diatomaceous earth. Determination of FXI and FXIa revealed a content of 0.05 IU FXI/ml and 3.5 mU FXIa/ml.

Tables 1-3 represent analytical results of samples before and after chromatography wherein runs 2-5 were performed with a reduced load of starting material for the heparin gel compared to run 1.

TABLE 1
Analysis of starting material.

| Sample A - Starting | | Reduced column load | | | |
|---|---|---|---|---|---|
| material | run 1 | run 2 | run 3 | run 4 | run 5 |
| IgG [g/L] | 7.12 | 6.94 | 6.65 | 5.47 | 7.02 |
| Factor XI [IU/mL] | 1.03 | 1.00 | 1.00 | 0.94 | 0.92 |
| Factor XIa [mU/mL] | 2.5 | 1.6 | 1.6 | 1.9 | 1.6 |

TABLE 2
Sample analysis of several experiments according to examples 1 (run 1,
run 4 and run 5) and 2 (run 2 and run 3).

| Sample B - after | | | | | |
|---|---|---|---|---|---|
| Heparin Sepharose | | Reduced column load | | | |
| Chrom. | run 1 | run 2 | run 3 | run 4 | run 5 |
| IgG [g/L] | 6.42 | 6.5 | 6.38 | 5.66 | 6.95 |
| Factor XI [IU/mL] | 0.05 | <0.01 | <0.01 | <0.01 | <0.01 |
| Factor XIa [mU/mL] | 3.5 | <1.0 | <1.0 | <1.0 | <1.0 |

TABLE 3
Sample analysis of several experiments according to example 1

| Sample C - after | | | | | |
|---|---|---|---|---|---|
| Heparin Sepharose | | Reduced column load | | | |
| and Hyflo treatment | run 1 | run 2 | run 3 | run 4 | run 5 |
| IgG [g/L] | 4.79 | — | — | 4.98 | 4.43 |
| Factor XI [IU/mL] | 0.02 | — | — | <0.01 | <0.01 |
| Factor XIa [mU/mL] | <1.0 | — | — | <1.0 | <1.0 |

1) A process for reduction and/or removal of FXI and FXIa from a source solution containing said coagulation factors and as main components immunoglobulins comprising the following steps:
a) contacting the FXI and/or FXIa containing solution with an affinity chromatographic gel wherein heparin or heparan is linked to the matrix material;
b) allowing adsorption of FXI and/or FXIa and
c) separation of the liquid deprived of FXI and/or FXIa from the adsorption media.

2) The process according to claim 1 wherein the active sites of the affinity chromatography gel are either already saturated or are allowed to saturate with antithrombin.

3) The process according to claim 1 wherein silicates selected from the group of silica, perlites, zeolithes or diatomaceous earth are additionally used as adsorbens of FXI and/or FXIa.

4) The process according to claim 1 wherein an additional adsorption medium selected from the group of aluminium hydroxide, aluminium oxide hydroxide or aluminium oxide is used.

5) The process according to claim 1 wherein one adsorption on heparin or heparan linked to a matrix material is performed after the chromatographic material was preconditioned with Antithrombin-III.

6) The process according to claim 1 wherein the source solution primarily contains immunoglobulin-γ.

7) The process according to claim 1 wherein the liquid deprived of FXI and/or FXIa is further subjected to at least one virus inactivation step selected from solvent/detergent treatment, UV-radiation, pasteurization, low pH incubation, caprylate precipitation or nanofiltration to obtain a virus inactivated solution deprived of FXI and/or FXIa.

8) The process according to claim 1 wherein the, optionally virus inactivated, liquid deprived of FXI and/or FXIa is concentrated to obtain a concentrate of a pharmaceutically active component, which is optionally formulated to obtain a pharmaceutical composition.

9) The process according to claim 1 wherein an immunoglobulin-y containing composition is applied to a heparin-affinity chromatography resin in a loading buffer of 10-18 mS conductivity;
(ii) followed by washing of the resin with a buffer of 10-18 mS conductivity and combination of the flow-through of step (i) and the effluent of the washing procedure;
(iii) performing at least one virus inactivation step employing tri-N-butyl phospate and a detergent;
(iv) followed by a concentrating step;
(v) optionally formulation of the obtained immunoglobulin-y containing solution.

10) A concentrate of a pharmaceutically active component obtainable by the process of claim 9.

11) A pharmaceutical composition obtainable from the concentrate of claim 10.
